(19) United States
(12) Patent Application Publication
(10) Pub. No.: US 2025/0386805 A1
(43) Pub. Date: Dec. 25, 2025

(54) ANIMAL WEARABLE DEVICE NOTIFICATION SYSTEM AND METHOD

(71) Applicant: MyAnIML Inc., Overland Park, KS (US)

(72) Inventors: SHEKHAR GUPTA, OVERLAND PARK, KS (US); NATHAN LEIKER, HAYS, KS (US); ABHIMANYU GUPTA, OVERLAND PARK, KS (US)

(21) Appl. No.: 18/752,277

(22) Filed: Jun. 24, 2024

(51) Int. Cl. A01K 29/00; G06N 20/00; G16H 50/30

(52) U.S. Cl. CPC A01K 29/005; G06N 20/00; G16H 50/30

(57) ABSTRACT

A wearable computing device includes a memory storing computer-readable instructions and at least one processor to execute the instructions to obtain movement information associated with an animal wearing the wearable computing device, compare the movement information with at least one machine learning model, each machine learning model related to a particular animal health condition, determine that the movement information indicates a potential health condition for the animal wearing the wearable computing device, obtain animal identification information for the animal with the potential health condition, and transmit a notification indicating the animal identification information for the animal with the potential health condition.

### BACKGROUND

Animals such as cattle suffer from a variety of different diseases and ailments. As an example, Bovine Respiratory Disease Complex (BRDC) is a disease complex that includes shipping fever, pneumonia, diarrhea, and can be viral or bacterial. Bovine respiratory disease (BRD) has a significant economic impact on the U.S. cattle industry resulting in losses of $900 million to $1 billion annually. BRD accounts for 50% to 70% of all deaths in feedlot cattle, resulting in direct losses of animals. BRD decreases average daily gain in cattle, leading to longer days on feed and increases costs. BRD can negatively impact hot carcass weight, marbling scores, and overall carcass quality grades, reducing carcass value. According to research, cattle treated once, twice, or three or more times for BRD see net returns decrease by $38, $167, and $230 per calf, respectively, due to performance losses. The USDA estimates that 16% of cattle in large feedlots are affected by BRD, costing $23.60 per case on average. However, other research points to even higher costs. Hence, BRD remains the costliest disease impacting the U.S. cattle industry through significant treatment expenditures, mortality losses, reduced efficiencies, diminished carcass quality, and ultimately lower profitability for producers.

It is with these issues in mind, among others, that various aspects of the disclosure were conceived.

### SUMMARY

The present disclosure is directed to an animal wearable device notification system and method. In one example, the system may include a plurality of wearable devices that may be worn by an animal, a client computing device, and a server computing device. A wearable device may execute an animal monitoring application that may determine that an animal is suffering from a potential health condition and may send a notification to the client computing device and/or the server computing device. The notification may include information associated with the animal such as animal identification information or location information of the animal. In addition, the notification may include information associated with the potential health condition such as an identification of the potential health condition. In one example, the notification may be provided by one or more light devices of the wearable computing device.

In one example, a wearable computing device may include a memory storing computer-readable instructions and at least one processor to execute the instructions to obtain movement information associated with an animal wearing the wearable computing device, compare the movement information with at least one machine learning model, each machine learning model related to a particular animal health condition, determine that the movement information indicates a potential health condition for the animal wearing the wearable computing device, obtain animal identification information for the animal with the potential health condition, and transmit a notification indicating the animal identification information for the animal with the potential health condition.

In another example, a method may include obtaining, by a computing device, movement information associated with an animal wearing the computing device, comparing, by the computing device, the movement information with at least one machine learning model, each machine learning model related to a particular animal health condition, determining, by the computing device, that the movement information indicates a potential health condition for the animal wearing the computing device, obtaining, by the computing device, animal identification information for the animal with the potential health condition, and transmitting, by the computing device, a notification indicating the animal identification information for the animal with the potential health condition.

In another example, a non-transitory computer-readable storage medium includes instructions stored thereon that, when executed by a computing device cause the computing device to perform operations, the operations including obtaining movement information associated with an animal wearing the computing device, comparing the movement information with at least one machine learning model, each machine learning model related to a particular animal health condition, determining that the movement information indicates a potential health condition for the animal wearing the computing device, obtaining animal identification information for the animal with the potential health condition, and transmitting a notification indicating the animal identification information for the animal with the potential health condition.

These and other aspects, features, and benefits of the present disclosure will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a block diagram of an animal wearable device notification system according to an example of the instant disclosure.

FIG. 2 is a block diagram of an animal wearable computing device according to an example of the instant disclosure.

FIG. 3 is a flowchart of a method of transmitting a notification indicating animal identification information for an animal with a potential health condition according to an example of the instant disclosure.

FIG. 4 shows an example of a system for implementing certain aspects of the present technology.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Aspects of an animal wearable device notification system includes a plurality of wearable devices that may be worn by an animal, at least one client computing device, and at least one server computing device. A wearable device may execute an animal monitoring application that may determine that an animal is suffering from a potential health condition and may send a notification to the at least one client computing device and/or the at least one server computing device. The notification may include information associated with the animal such as animal identification information or location information of the animal. In addition, the notification may include information associated with the potential health condition such as an identification of the potential health condition. In one example, the notification may be provided by one or more light devices of the wearable computing device.

As an example, a wearable computing device may include a memory storing computer-readable instructions and at least one processor to execute the instructions to obtain movement information associated with an animal wearing the wearable computing device, compare the movement information with at least one machine learning model, each machine learning model related to a particular animal health condition, determine that the movement information indicates a potential health condition for the animal wearing the wearable computing device, obtain animal identification information for the animal with the potential health condition, and transmit a notification indicating the animal identification information for the animal with the potential health condition.

The animal wearable device notification system may include at least one wearable computing device that may be worn by an animal such as a cow or a bull. The wearable computing device may be an ear tag that may send one or more notifications indicating a condition of the animal. In one example, the wearable computing device may be an Internet of Things (IoT) device such as the ear tag that may include one or more light devices such as one or more light emitting diodes (LEDs). The wearable computing device may have one or more sensor devices such as one or more accelerometers and one or more gyroscopes to obtain movement information associated with the animal.

In another example, the wearable computing device may be a nose tag that may be worn on a nose of the animal such as a nose of a bull or a cow. In another example, the wearable computing device may be worn on a collar of the animal.

The wearable computing device may function as a pedometer and detect a number of steps taken by the animal and may include one or more thermometers to determine a temperature of the animal.

In another example, the wearable computing device may have one or more microphones to obtain audio information about the animal and one or more speaker devices to provide audio output information about the animal.

In another example, the wearable computing device may include a global positioning system (GPS) device and a Bluetooth device.

In an example, the wearable computing device may provide output information using the one or more light devices based on a condition of the animal. As an example, when the wearable computing device determines lameness, the one or more light devices may provide a particular color such as red. In another example, when the wearable computing device determines Bovine Respiratory Disease Complex (BRDC), the one or more light devices may provide a different color. If the animal is not moving, the one or more light devices may provide another color and could send information such location information of the animal using the GPS device. It may be possible that the animal is deceased or injured and the animal may need medical attention.

In another example, the one or more light devices and/or the one or more speakers may provide information associated with the condition of the animal. In some situations, the wearable computing device may provide information associated with an environment of the animal such as information associated with a temperature of the environment. For example, if the temperature is above a particular temperature, the one or more light devices or the one or more speakers may provide information about the temperature. In another example, if the temperature is below a particular temperature, the one or more light devices or the one or more speakers may provide information about the temperature.

In one example, the wearable computing device may be attached or installed to an ear tag to be worn by cattle. The wearable computing device may be powered by one or more batteries and/or solar power and/or wind power. In another example, the wearable computing device may be powered by kinetic energy of the animal such as movement of ears of the animal. If the one or more batteries have less than a particular level of energy, the wearable computing device may send a notification.

For example, in BRD, early symptoms include difficulty breathing and cough. However, the cough can be due to various reasons such as an object stuck in a throat of the animal. When an object becomes stuck, it may be dissolved on its own or a pen rider may assist and unclog the object. However, if BRD is present, it may be difficult to determine. By the time an animal suffers a fever, it may be too late. If a pen rider determines that a cow has a fever, the cow may be sick and ready for hospitalization.

The one or more sensor devices such as the one or more accelerometers and the one or more gyroscopes may monitor for changes in the head of the animal such as a sudden movement of the head. If the wearable computing device is worn on a head of the animal such as ears, when the animal coughs or there is a breathing issue, the wearable computing device may receive movement information using the one or more accelerometers and the one or more gyroscopes. A cough may cause the animal to move their head very violently and in a different way from when the animal is eating or swallowing. The wearable computing device may determine that the animal is coughing and may also determine whether the animal is suffering from a persistent cough or suffering from a less frequent cough or head movement. The wearable computing device also may determine whether the frequency of the cough is increasing or decreasing. The wearable computing device also may determine whether the animal is suffering from breathing issues. When an animal is having breathing issues, the animal may move their head around to figure out a better way to breathe.

The wearable computing device may determine that the animal is suffering from a potential health condition and may send a notification that may include location information of the animal or identification information of the animal. The notification may be sent to a client computing device such as a computing device of a person that may be monitoring the animal. In one example, the notification may be sent using a network such as a wireless network, a cellular network, or a Bluetooth network, among others.

The wearable computing device may determine a number of coughs that occur in a particular period of time. The wearable computing device may determine a first cough or a first breathing issue and start a time counter and determine how many coughs or breathing issues occur in the particular period of time or duration. The number of coughs in the particular period of time may be sent in a notification to a person monitoring the animal that may include a veterinarian.

The wearable computing device may determine a cough based on the movement information using artificial intelligence (AI) models. In one example, the wearable computing device may use machine learning and deep learning techniques that may be trained to recognize motion patterns of an animal when a cough occurs. The models may achieve high accuracy, recall, precision, and F1 scores in recognizing coughs. Thus, the wearable computing device may determine a number of potential health conditions of an animal such as respiratory diseases and BRD, among others. As an example, BRD may affect young calves that are coming to a feedlot for a first time. Although BRD is regional to the United States, there are other potential health conditions in other parts of the world. The wearable computing device may be trained to determine any number of symptoms to determine potential health conditions of the animal.

In another example, the wearable computing device may have a methane sensor. The methane sensor may be used to assist in tracking an individual cow's contribution to greenhouse gas emissions, allowing for targeted interventions to reduce their environmental impact.

In another example, the wearable computing device may include a biocompatible rumen patch for antibiotic delivery. This could deliver targeted antibiotics directly to the site of infection, reducing overall antibiotic use and promoting herd health.

In another example, the wearable computing device may have AI-powered fall detection. This could allow for faster intervention in case of injury or calving issues.

With GPS, the wearable computing device may be able to identify where the cow spends most of its time. So for example, if a cow is grazing grass on a large field in one location and then suddenly moves to another location, it is possible that there is no grass available. Alternatively, some structure may have blocked the path of the animal. The pen rider can be sent a message or an alert and may check out an associated area.

In one example, the animal wearable device notification system may build a number of artificial intelligence models that may be used to determine potential health conditions. The animal wearable device notification system may obtain a large dataset including movement information of animals such as cattle. The movement information may indicate healthy movement information and may include potential health conditions such as movement of a head of an animal related to coughing. Ranchers and veterinarians may be used to label data in the dataset as healthy movement and potential health conditions. The models may be trained. The annotated dataset may be split into a training dataset and a validation dataset. This may include training a selected deep learning model on the training dataset using techniques such as transfer learning or data augmentation to improve performance. Model evaluation may include evaluating the trained model performance on the validation dataset to measure metrics such as accuracy, precision, recall, and F1-score for detecting head jerks due to cough or breathing issues. Model optimization may include improving the model's performance when it is unsatisfactory by using techniques such as hyperparameter tuning, architecture modifications, or additional data collection and annotation.

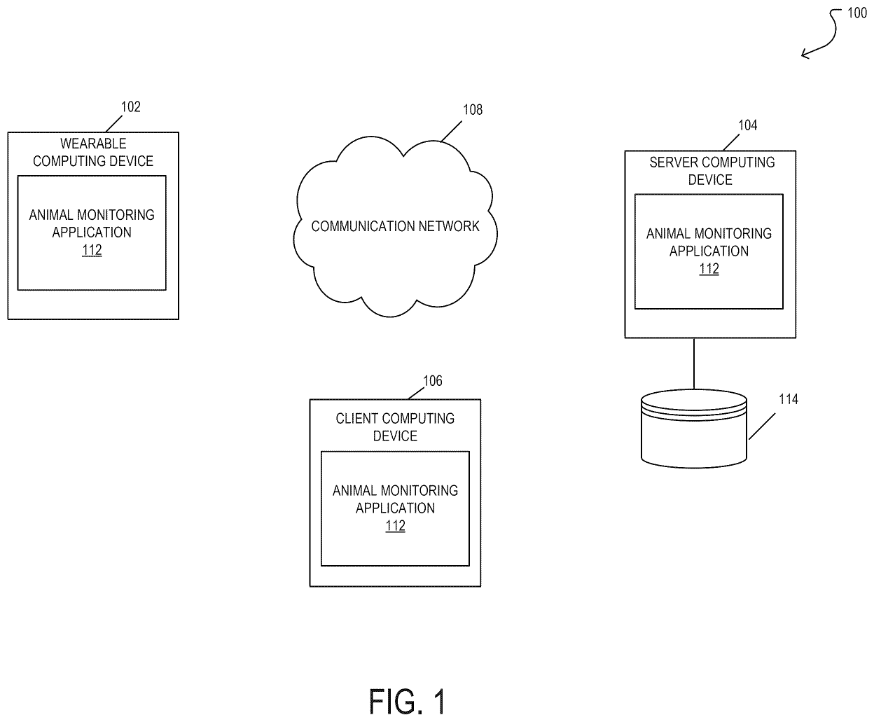

FIG. 1 is a block diagram of an animal wearable device notification system 100 according to an example of the instant disclosure. The system may include a plurality of wearable computing devices 102. Each wearable computing device 102 may be worn by an animal such as cattle or a bovine including cows and bulls. In one example, the wearable computing device 102 may be worn on an ear tag of the animal, a nose tag of the animal, a collar of the animal, or in another location. However, in other examples, the wearable computing device 102 may be worn by animals including livestock, domesticated animals, or wild animals. The system 100 may include at least one server computing device 104 and at least one client computing device 106. The at least one server computing device 104 may have or be in communication with at least one database 114.

The wearable computing device 102, the client computing device 106, and the server computing device 104 may have an animal monitoring application 112 that may be a component of an application and/or service executable by the wearable computing device 102, the at least one client computing device 106, and/or the server computing device 104. For example, the animal monitoring application 112 may be a single unit of deployable executable code or a plurality of units of deployable executable code. According to one aspect, the animal monitoring application 112 may include one component that may be a web application, a native application, and/or a mobile application (e.g., an app) downloaded from a digital distribution application platform that allows users to browse and download applications developed with mobile software development kits (SDKs) including the App Store and GOOGLE PLAY®, among others.

The animal wearable device notification system 100 also may include a relational database management system (RDBMS) or another type of database management system such as a NoSQL database system that stores and communicates data from at least one database 114. The data stored in the database 114 may be associated with the plurality of animals such as information associated with each of the plurality of animals and machine learning models associated with health conditions of the animals.

The at least one wearable computing device 102, the at least one client computing device 106, and the at least one server computing device 104 may be configured to receive data from and/or transmit data through a communication network 108. Although the wearable computing device 102, the client computing device 106, and the server computing device 104 are shown as a single computing device, it is contemplated each computing device may include multiple computing devices.

The communication network 108 can be the Internet, an intranet, or another wired or wireless communication network. For example, the communication network may include a Mobile Communications (GSM) network, a code division multiple access (CDMA) network, 3^{rd} Generation Partnership Project (GPP) network, an Internet Protocol (IP) network, a wireless application protocol (WAP) network, a WiFi network, a Bluetooth network, a near field communication (NFC) network, a LoRaWAN network, a satellite communications network, or an IEEE 802.11 standards network, as well as various communications thereof. Other conventional and/or later developed wired and wireless networks may also be used.

The wearable computing device 102 may include at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions. In addition, the wearable computing device 102 further includes at least one communications interface to transmit and receive communications, messages, and/or signals.

The client computing device 106 may include at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions. In addition, the client computing device 106 further includes at least one communications interface to transmit and receive communications, messages, and/or signals.

The client computing device 106 could be a programmable logic controller, a programmable controller, a laptop computer, a smartphone, a personal digital assistant, a tablet computer, a standard personal computer, or another processing device. The client computing device 106 may include a display, such as a computer monitor, for displaying data and/or graphical user interfaces. The client computing device 106 may also include a Global Positioning System (GPS) hardware device for determining a particular location, an input device, such as one or more cameras or imaging devices, a keyboard or a pointing device (e.g., a mouse, trackball, pen, or touch screen) to enter data into or interact with graphical and/or other types of user interfaces. In an exemplary embodiment, the display and the input device may be incorporated together as a touch screen of the smartphone or tablet computer.

The server computing device 104 may include at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions. In addition, the server computing device 104 further includes at least one communications interface to transmit and receive communications, messages, and/or signals.

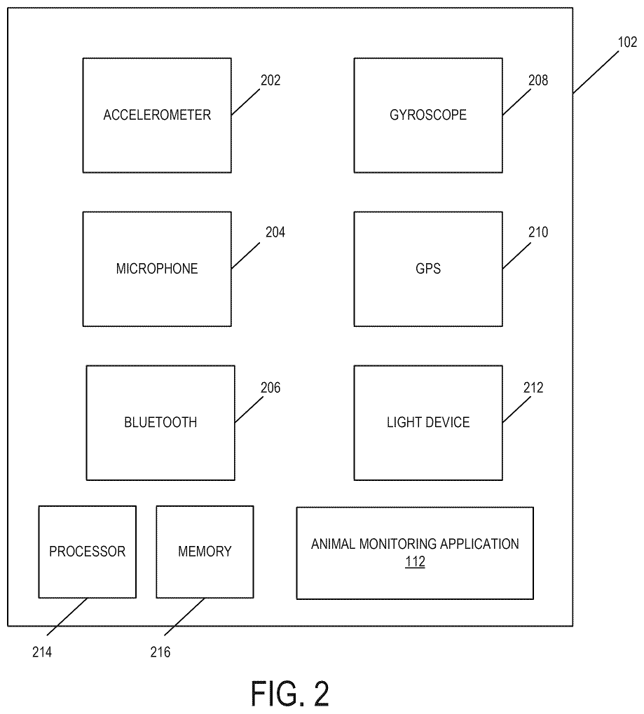

FIG. 2 is a block diagram of an animal wearable computing device 102 according to an example of the instant disclosure. As shown in FIG. 2, the animal wearable computing device may include at least one processor 214 and memory 216. The animal wearable computing device 102 may include one or more accelerometers 202, one or more microphones 204 to obtain audio information associated with the animal, one or more Bluetooth hardware devices 206, one or more gyroscopes 208, one or more GPS hardware devices 210 to obtain location information of the wearable computing device 102, and one or more light devices 212 such as one or more light emitting diodes (LEDs), among other sensors such as thermometers and hardware devices. The animal monitoring application 112 may be in a computer-readable storage medium of the animal wearable computing device 112 and executed by the processor 214.

In a further example, the animal wearable computing device 102 may include a methane sensor. The methane sensor may be used to assist in tracking an individual cow's contribution to greenhouse gas emissions, allowing for targeted interventions to reduce their environmental impact.

In another example, the animal wearable computing device 102 may include a biocompatible rumen patch for antibiotic delivery. This could deliver targeted antibiotics directly to the site of infection, reducing overall antibiotic use and promoting herd health.

In another example, the animal wearable computing device 102 may have AI-powered fall detection. This could allow for faster intervention in case of injury or calving issues.

With GPS, the animal wearable computing device 102 may be able to identify where the cow spends most of its time. So for example, if a cow is grazing grass on a large field in one location and then suddenly moves to another location, it is possible that there is no grass available. Alternatively, some structure may have blocked the path of the animal. The pen rider can be sent a message or an alert and may check out an associated area.

In another example, the animal wearable computing device 102 may include one or more weight sensors to determine and measure weight of the animal. As an animal gets sick, it may lose weight. The one or more weight sensors may be attached to or placed on one or more legs or feet of the animal. If the weight sensor is attached to a foot of the animal, the weight sensor also may be able to detect that the animal is not placing weight or is placing weight on a particular leg or foot. This may be used to determine an injury to a leg or foot or may indicate that the animal is suffering from a health condition or disease.

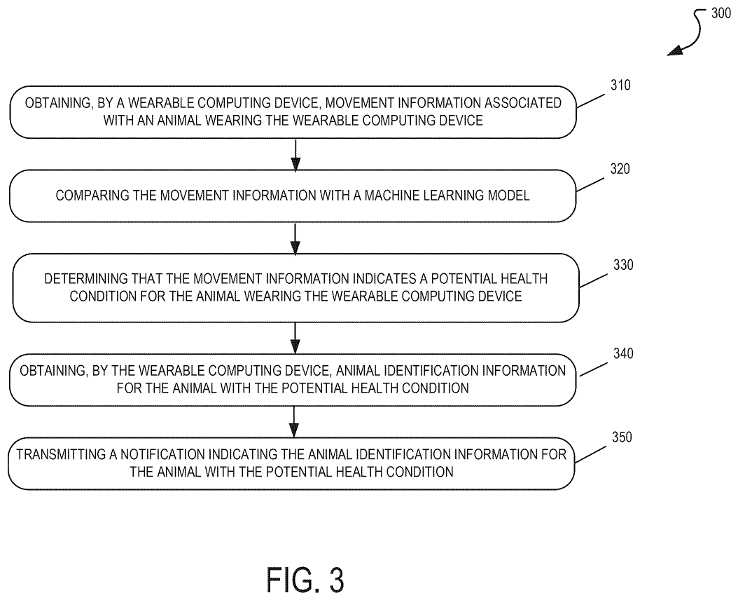

FIG. 3 illustrates an example method 300 of transmitting a notification indicating animal identification information for an animal with a potential health condition according to an example of the instant disclosure. Although the example method 300 depicts a particular sequence of operations, the sequence may be altered without departing from the scope of the present disclosure. For example, some of the operations depicted may be performed in parallel or in a different sequence that does not materially affect the function of the method 300. In other examples, different components of an example device or system that implements the method 300 may perform functions at substantially the same time or in a specific sequence.

According to some examples, the method 300 may include obtaining movement information associated with an animal wearing the wearable computing device 102 at block 310. As an example, the movement information may be obtained from at least one accelerometer 202 of the wearable computing device 102. In another example, the movement information may be obtained from the movement information is obtained from at least one gyroscope 208 of the wearable computing device 102. As an example, the wearable computing device 102 may be attached to one of an ear tag of the animal, a nose tag of the animal, and a collar of the animal.

As an example, the particular animal health condition may be one of lameness, death, and Bovine Respiratory Disease Complex (BRDC), among others. As an example, the animal may be a bovine such as a cow or a bull.

Next, according to some examples, the method 300 may include comparing the movement information with at least one machine learning model, each machine learning model related to a particular animal health condition at block 320. As an example, a first machine learning model may be related to BRDC, a second machine learning model may be related to lameness, etc.

Next, according to some examples, the method 300 may include determining that the movement information indicates a potential health condition for the animal wearing the wearable computing device 102 at block 330.

Next, according to some examples, the method 300 may include obtaining animal identification information for the animal with the potential health condition at block 340.

Next, according to some examples, the method 300 may include transmitting a notification indicating the animal identification information for the animal with the potential health condition at block 350.

According to some examples, the method 300 may include illuminating at least one light 212 of the wearable computing device, the at least one light indicating the potential health condition. As an example, the at least one light is a color that indicates one of a respiratory disease and Bovine Respiratory Disease Complex (BRDC).

According to some examples, the method 300 may include determining that the movement information indicates a violent movement of a head of the animal representative of a cough.

According to some examples, the method 300 may include determining that the movement information indicates a plurality of violent movements of a head of the animal representative of a number of coughs in a particular period of time.

According to some examples, the method 300 may include determining that the movement information indicates a plurality of violent movements of a head of the animal representative of a number of coughs in a first particular period of time and determining that the movement information indicates a plurality of violent movements of a head of the animal representative of a number of coughs in a second particular period of time and determining that the number of coughs in the first particular period of time is greater than the number of coughs in the second particular period of time. This may indicate a worsening of a health of the animal.

According to some examples, the method 300 may include determining that the movement information indicates a plurality of violent movements of a head of the animal representative of a number of coughs in a first particular period of time and determining that the movement information indicates a plurality of violent movements of a head of the animal representative of a number of coughs in a second particular period of time and determining that the number of coughs in the first particular period of time is less than the number of coughs in the second particular period of time. This may indicate an improvement of a health of the animal.

According to some examples, the method 300 may include determining one of a temperature of the animal and a temperature of an environment of the animal.

According to some examples, the method 300 may include determining that the movement information indicates that the animal has not moved for a particular period of time.

According to some examples, the method 300 may include providing audio output information related to the potential health condition of the animal.

As an example, the wearable computing device 102 may be powered by one of one or more batteries, solar power, wind power, and kinetic energy provided by the animal.

According to some examples, the method 300 may include determining location information of the animal using a global positioning system (GPS) hardware device 210 of the wearable computing device 102.

According to some examples, the method 300 may include sending the notification to a client computing device 106 of one of a pen rider and a veterinarian.

According to some examples, the wearable computing device 102 may communicates with a biocompatible rumen patch for antibiotic delivery to deliver targeted antibiotics directly to a site of infection of the animal.

According to some examples, the wearable computing device 102 may include one or more weight sensors to determine and measure a weight of the animal at a first time and determine and measure the weight of the animal at a second time and determine that there is the potential health condition based on a change in the weight of the animal at the first time and the weight of the animal at a second time.

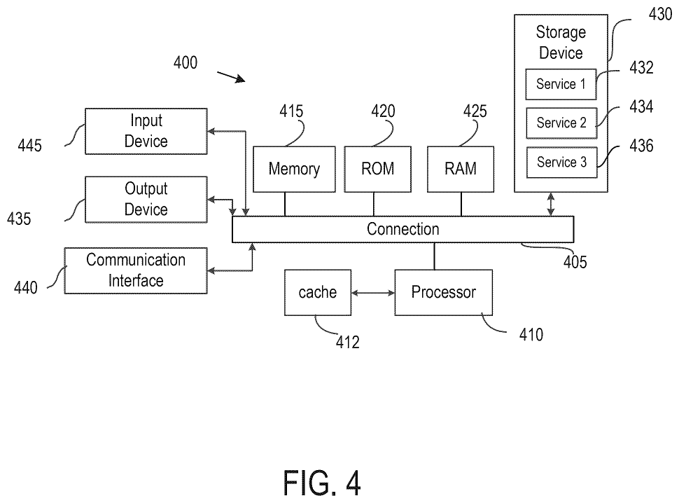

FIG. 4 shows an example of computing system 400, which can be for example any computing device making up the computing device such as the wearable computing device 102, the client computing device 106, the server computing device 104, or any component thereof in which the components of the system are in communication with each other using connection 405. Connection 405 can be a physical connection via a bus, or a direct connection into processor 410, such as in a chipset architecture. Connection 405 can also be a virtual connection, networked connection, or logical connection.

In some embodiments, computing system 400 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 400 includes at least one processing unit (CPU or processor) 410 and connection 405 that couples various system components including system memory 415, such as read-only memory (ROM) 420 and random access memory (RAM) 425 to processor 410. Computing system 400 can include a cache of high-speed memory 412 connected directly with, in close proximity to, or integrated as part of processor 410.

Processor 410 can include any general purpose processor and a hardware service or software service, such as services 432, 434, and 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction, computing system 400 includes an input device 445, which can represent any number of input mechanisms, such as a microphone for speech, a touch-sensitive screen for gesture or graphical input, keyboard, mouse, motion input, speech, etc. Computing system 400 can also include output device 435, which can be one or more of a number of output mechanisms known to those of skill in the art. In some instances, multimodal systems can enable a user to provide multiple types of input/output to communicate with computing system 400. Computing system 400 can include communications interface 440, which can generally govern and manage the user input and system output. There is no restriction on operating on any particular hardware arrangement, and therefore the basic features here may easily be substituted for improved hardware or firmware arrangements as they are developed.

Storage device 430 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 430 can include software services, servers, services, etc., that when the code that defines such software is executed by the processor 410, it causes the system to perform a function. In some embodiments, a hardware service that performs a particular function can include the software component stored in a computer-readable medium in connection with the necessary hardware components, such as processor 410, connection 405, output device 435, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Illustrative examples of the disclosure include:

Aspect 1: A wearable computing device comprising: a memory storing computer-readable instructions and at least one processor to execute the instructions to obtain movement information associated with an animal wearing the wearable computing device, compare the movement information with at least one machine learning model, each machine learning model related to a particular animal health condition, determine that the movement information indicates a potential health condition for the animal wearing the wearable computing device, obtain animal identification information for the animal with the potential health condition, and transmit a notification indicating the animal identification information for the animal with the potential health condition.

Aspect 2: The wearable computing device of Aspect 1, wherein the movement information is obtained from at least one accelerometer of the wearable computing device and at least one gyroscope of the wearable computing device.

Aspect 3: The wearable computing device of Aspects 1 and 2, wherein the wearable computing device communicates with a biocompatible rumen patch for antibiotic delivery to deliver targeted antibiotics directly to a site of infection of the animal.

Aspect 4: The wearable computing device of Aspects 1 to 3, the at least one processor further to execute the instructions to illuminate at least one light of the wearable computing device, the at least one light indicating the potential health condition and the at least one light is a color that indicates one of a respiratory disease and Bovine Respiratory Disease Complex (BRDC.

Aspect 5: The wearable computing device of Aspects 1 to 4, wherein the wearable computing device comprises one or more weight sensors to determine and measure a weight of the animal at a first time and determine and measure the weight of the animal at a second time and determine that there is the potential health condition based on a change in the weight of the animal at the first time and the weight of the animal at a second time

Aspect 6: The wearable computing device of Aspects 1 to 5, the at least one processor further to execute the instructions to determine that the movement information indicates a violent movement of a head of the animal representative of a cough.

Aspect 7: The wearable computing device of Aspects 1 to 6, the at least one processor further to execute the instructions to determine that the movement information indicates a plurality of violent movements of a head of the animal representative of a number of coughs in a particular period of time.

Aspect 8: The wearable computing device of Aspects 1 to 7, the at least one processor further to execute the instructions to determine that the movement information indicates a plurality of violent movements of a head of the animal representative of a number of coughs in a first particular period of time and determine that the movement information indicates a plurality of violent movements of a head of the animal representative of a number of coughs in a second particular period of time and determine that the number of coughs in the first particular period of time is greater than the number of coughs in the second particular period of time.

Aspect 9: The wearable computing device of Aspects 1 to 8, the at least one processor further to execute the instructions to determine that the movement information indicates a plurality of violent movements of a head of the animal representative of a number of coughs in a first particular period of time and determine that the movement information indicates a plurality of violent movements of a head of the animal representative of a number of coughs in a second particular period of time and determine that the number of coughs in the first particular period of time is less than the number of coughs in the second particular period of time.

Aspect 10: The wearable computing device of Aspects 1 to 9, the at least one processor further to execute the instructions to determine one of a temperature of the animal and a temperature of an environment of the animal.

Aspect 11: The wearable computing device of Aspects 1 to 10, the at least one processor further to execute the instructions to determine that the movement information indicates that the animal has not moved for a particular period of time.

Aspect 12: The wearable computing device of Aspects 1 to 11, wherein the wearable computing device is attached to one of an ear tag, a nose tag, and a collar.

Aspect 13: The wearable computing device of Aspects 1 to 12, the at least one processor further to execute the instructions to provide audio output information related to the potential health condition of the animal.

Aspect 14: The wearable computing device of Aspects 1 to 13, wherein the wearable computing device is powered by one of one or more batteries, solar power, wind power, and kinetic energy provided by the animal.

Aspect 15: The wearable computing device of Aspects 1 to 14, wherein the particular animal health condition comprises one of lameness, death, and Bovine Respiratory Disease Complex (BRDC).

Aspect 16: The wearable computing device of Aspects 1 to 15, the at least one processor further to execute the instructions to determine location information of the animal using a global positioning system (GPS) hardware device of the wearable computing device.

Aspect 17: The wearable computing device of Aspects 1 to 16, the at least one processor further to execute the instructions to send the notification to a computing device of one of a pen rider and a veterinarian.

Aspect 18: The wearable computing device of Aspects 1 to 17, wherein the animal comprises a bovine.

Aspect 19: A method comprising obtaining, by a computing device, movement information associated with an animal wearing the computing device, comparing, by the computing device, the movement information with at least one machine learning model, each machine learning model related to a particular animal health condition, determining, by the computing device, that the movement information indicates a potential health condition for the animal wearing the computing device, obtaining, by the computing device, animal identification information for the animal with the potential health condition, and transmitting, by the computing device, a notification indicating the animal identification information for the animal with the potential health condition.

Aspect 20: A non-transitory computer-readable storage medium comprising instructions stored thereon that, when executed by a computing device cause the computing device to perform operations, the operations comprising obtaining movement information associated with an animal wearing the computing device, comparing the movement information with at least one machine learning model, each machine learning model related to a particular animal health condition, determining that the movement information indicates a potential health condition for the animal wearing the computing device, obtaining animal identification information for the animal with the potential health condition, and transmitting a notification indicating the animal identification information for the animal with the potential health condition.

What is claimed is:

1. A wearable computing device comprising:
a memory storing computer-readable instructions; and
at least one processor to execute the instructions to:
obtain movement information associated with an animal wearing the wearable computing device;
compare the movement information with at least one machine learning model, each machine learning model related to a particular animal health condition;
determine that the movement information indicates a potential health condition for the animal wearing the wearable computing device;
obtain animal identification information for the animal with the potential health condition; and
transmit a notification indicating the animal identification information for the animal with the potential health condition.

2. The wearable computing device of claim 1, wherein the movement information is obtained from at least one accelerometer of the wearable computing device and at least one gyroscope of the wearable computing device.

3. The wearable computing device of claim 1, wherein the wearable computing device communicates with a biocompatible rumen patch for antibiotic delivery to deliver targeted antibiotics directly to a site of infection of the animal.

4. The wearable computing device of claim 1, the at least one processor to execute the instructions to illuminate at least one light of the wearable computing device, the at least one light indicating the potential health condition and the at least one light is a color that indicates one of a respiratory disease and Bovine Respiratory Disease Complex (BRDC).

5. The wearable computing device of claim 1, wherein the wearable computing device comprises one or more weight sensors to determine and measure a weight of the animal at a first time and determine and measure the weight of the animal at a second time and determine that there is the potential health condition based on a change in the weight of the animal at the first time and the weight of the animal at a second time.

6. The wearable computing device of claim 1, the at least one processor further to execute the instructions to determine that the movement information indicates a violent movement of a head of the animal representative of a cough.

7. The wearable computing device of claim 1, the at least one processor further to execute the instructions to determine that the movement information indicates a plurality of violent movements of a head of the animal representative of a number of coughs in a particular period of time.

8. The wearable computing device of claim 1, the at least one processor further to execute the instructions to determine that the movement information indicates a plurality of violent movements of a head of the animal representative of a number of coughs in a first particular period of time and determine that the movement information indicates a plurality of violent movements of a head of the animal representative of a number of coughs in a second particular period of time and determine that the number of coughs in the first particular period of time is greater than the number of coughs in the second particular period of time.

9. The wearable computing device of claim 1, the at least one processor further to execute the instructions to determine that the movement information indicates a plurality of violent movements of a head of the animal representative of a number of coughs in a first particular period of time and determine that the movement information indicates a plurality of violent movements of a head of the animal representative of a number of coughs in a second particular period of time and determine that the number of coughs in the first particular period of time is less than the number of coughs in the second particular period of time.

10. The wearable computing device of claim 1, the at least one processor further to execute the instructions to determine one of a temperature of the animal and a temperature of an environment of the animal.

11. The wearable computing device of claim 1, the at least one processor further to execute the instructions to determine that the movement information indicates that the animal has not moved for a particular period of time.

12. The wearable computing device of claim 1, wherein the wearable computing device is attached to one of an ear tag, a nose tag, and a collar.

13. The wearable computing device of claim 1, the at least one processor further to execute the instructions to provide audio output information related to the potential health condition of the animal.

14. The wearable computing device of claim 1, wherein the wearable computing device is powered by one of one or more batteries, solar power, wind power, and kinetic energy provided by the animal.

15. The wearable computing device of claim 1, wherein the particular animal health condition comprises one of lameness, death, and Bovine Respiratory Disease Complex (BRDC).

16. The wearable computing device of claim 1, the at least one processor further to execute the instructions to determine location information of the animal using a global positioning system (GPS) hardware device of the wearable computing device.

17. The wearable computing device of claim 1, the at least one processor further to execute the instructions to send the notification to a computing device of one of a pen rider and a veterinarian.

18. The wearable computing device of claim 1, wherein the animal comprises a bovine.

19. A method, comprising:
obtaining, by a computing device, movement information associated with an animal wearing the computing device;
comparing, by the computing device, the movement information with at least one machine learning model, each machine learning model related to a particular animal health condition;
determining, by the computing device, that the movement information indicates a potential health condition for the animal wearing the computing device;
obtaining, by the computing device, animal identification information for the animal with the potential health condition; and
transmitting, by the computing device, a notification indicating the animal identification information for the animal with the potential health condition.

20. A non-transitory computer-readable storage medium comprising instructions stored thereon that, when executed by a computing device cause the computing device to perform operations, the operations comprising:
obtaining movement information associated with an animal wearing the computing device;
comparing the movement information with at least one machine learning model, each machine learning model related to a particular animal health condition;
determining that the movement information indicates a potential health condition for the animal wearing the computing device;
obtaining animal identification information for the animal with the potential health condition; and
transmitting a notification indicating the animal identification information for the animal with the potential health condition.
